(19) United States
(12) Patent Application Publication
(10) Pub. No.: US 2011/0152228 A1
(43) Pub. Date: Jun. 23, 2011

(54) SPRAYABLE PHARMACEUTICAL COMPOSITIONS COMPRISING A CORTICOID AND AN OILY PHASE

(75) Inventors: Nathalie Willcox, Saint Vallier de Thiey (FR); Sandrine Orsoni, Mandelieu (FR)

(73) Assignee: Galderma, S.A., Cham (CH)

(21) Appl. No.: 13/038,483

(22) Filed: Mar. 2, 2011

Related U.S. Application Data
(63) Continuation of application No. 12/128,975, filed on May 29, 2008, now abandoned.
Application No. 12/128,975 is a continuation of application No. PCT/FR2006/051259, filed on Nov. 30, 2006.

(30) Foreign Application Priority Data
Nov. 30, 2005 (FR) 05/12176

(51) Int. Cl. A61K 31/573; A61P 17/00; A61P 31/12; A61P 3/10; A61P 35/00; A61P 11/06; A61P 9/00

(52) U.S. Cl. 514/180; 514/179

(57) ABSTRACT

Anhydrous sprayable pharamecutical/dermatological compositions containing, as active pharmaceutical agent, a corticoid, preferably clobetasol propionate, and an oily phase, are formulated in a physiologically acceptable medium, and are useful for the treatment of a variety of conditions and afflictions, notably psoriasis.

### CROSS-REFERENCE TO EARLIER APPLICATIONS

This application is a continuation of U.S. application Ser. No. 12/128,975 filed May 29, 2008, which is a continuation of PCT/FR 2006/051259, filed Nov. 30, 2006, and designating the United States (published in the French language on Jun. 6, 2007 as WO 2007/063254 A1; the title and abstract were also published in English), and claiming priority under 35 U.S.C. §119 of FR 0512176, filed Nov. 30, 2005, each hereby expressly incorporated by reference in its entirety and each assigned to the assignee hereof.

### BACKGROUND OF THE INVENTION

1. Technical Field of the Invention

The present invention relates to sprayable anhydrous compositions containing a corticoid as an active pharmaceutical agent, preferably clobetasol propionate, and an oily phase, formulated into a physiologically acceptable medium, to methodology for preparing such compositions and to the administration thereof in dermatology.

2. Description of Background and/or Related and/or Prior Art

In the field of dermatology and of the formulation of pharmaceutical compositions, those skilled in the art seek compositions which not only should be physically and chemically stable, but also should make it possible to release the active agent and to promote the penetration of the latter through the layers of the skin in order to improve the effectiveness thereof.

The pharmaceutical composition should, in addition, have good cosmeticity and preferably be non-irritant.

There currently exist numerous topical compositions which contain a corticoid-type active agent and which make it possible to promote the penetration of said active agent into the skin through the presence, in particular, of a high content of propenetrating glycol. These compositions are formulated in the form of emulsions with a high content of fatty phase, which are commonly called “lipocreams”, in the form of anhydrous compositions which are called “ointments”, in the form of fluid compositions with a high content of volatile solvents, such as ethanol or isopropanol, for application to the scalp, also called “hair lotions”, or else in the form of viscous 0/W emulsions, which are also called “0/W creams”.

The stabilization of a formulation comprising such a percentage of glycol makes it necessary to include, in the emulsion, emulsifying stabilizing agents of the glycerol stearate or PEG 100 stearate type or else stabilizing agents or consistency factors of the white beeswax or cetostearyl alcohol type, which result in the formation of a viscous cream. This viscosity therefore makes the product difficult to apply. However, these compositions, firstly, are poorly accepted from a cosmetic point of view due to their viscosity and, secondly, exhibit risks of intolerance brought about by the presence of high proportions of glycol. Furthermore, these high viscosities make the formulations difficult to apply to the various parts of the body affected by the pathology. Consequently, most of the existing treatments, in the form of creams, gels or ointments, require the aid of a third person in order to apply them to the areas that are difficult to access. The third person must therefore touch both the product containing the active agent and the psoriatic plaques, thereby resulting in a situation which is not ideal from the point of view of comfortable use and safety for the third person. Those skilled in the art are also aware that non-compliance with the prescribed treatment for reasons mentioned above is one of the main causes of failure; the article “patients with psoriasis and their compliance with medication” (Richards et al., J. Am. Acad. Dermatol., Oct. 99, p 581-583) indicates that close to 40% of patients with a chronic disease such as psoriasis do not follow their treatment. It has been demonstrated that the patient's compliance with his or her treatment is directly linked to the characteristics of the vehicle of the composition applied. The article “Patients with psoriasis prefer solution and foam vehicles: a quantitative assessment of vehicle preference” (Housman et al., CUTIS, December 2002 vol. 70, p 327 to 332) indicates that psoriatic patients will prefer a solution or a foam rather than an ointment, a cream or a gel.

Those skilled in the art seek to improve these parameters.

In the prior art, the existing compositions often contain a high percentage of petroleum jelly in order to promote the occlusivity and the penetration of the active agent, but therefore have the drawback of being very greasy and tacky, and thus of not promoting comfortable and easy application. The other types of compositions commonly encountered in the prior art contain a high percentage of propenetrating glycerol in order to promote the penetration of the active agent, but are tacky and can cause problems of intolerance (“The critical role of the vehicle to therapeutic efficacy and patient compliance” Piacquadio et al., Journal of American Academy of Dermatology, August 1998). U.S. Pat. Nos. 5,972,920 and 6,579,512 describe sprayable compositions containing a high percentage of alcoholic and/or volatile solvents, and which are not of oily type. Although these compositions are easier to apply, they cannot provide the emollient effect desired for the patient's comfort.

### SUMMARY OF THE INVENTION

The present invention features physically and chemically stable anhydrous compositions containing clobetasol propionate useful for the treatment of psoriasis, such compositions being easy to use and having a cosmeticity which is acceptable for application to all the areas of the body that may be affected by the pathology. The term “composition containing clobetasol propionate” means any composition containing exclusively clobetasol propionate as bioactive ingredient. In particular, the composition contains no vitamin D derivative, and in particular no calcitriol.

According to the invention, the term “physical stability” means a composition which shows no modification to its macroscopic appearance (phase separation, change in color of appearance, etc.) nor to its microscopic appearance (recrystallization of active agents) after storage at temperatures of 25° C., 4° C. and 40° C., for 2, 4, 8, 12 weeks.

According to the invention, the term “chemical stability” means a composition in which the content of active ingredient remains stable after three months at ambient temperature and at 40° C. A stable content of active ingredient means, according to the invention, that the content shows very little variation relative to the initial content, i.e., the variation in content of active ingredient at time T should not be less than 90%, and more particularly than 95%, of the initial content at T0.

It has now surprisingly been found that a composition which is liquid at ambient temperature, and which is preferably sprayable, containing, formulated into a pharmaceutically acceptable vehicle:

a) a therapeutically effective amount of a corticoid in solubilized form, and more particularly clobetasol propionate;

b) an oily phase composed of one or more oils;

said composition containing no vitamin D derivative, provides a composition which avoids the above disadvantages and drawbacks.

### DETAILED DESCRIPTION OF BEST MODE AND SPECIFIC/PREFERRED EMBODIMENTS OF THE INVENTION

According to an advantageous embodiment of the invention, the corticosteroid is selected from the group consisting of betamethasone clobetasol, clobetasone, desoximethasone, diflucortolon, diflorasone, fluocinonide, flumethasone, fluocinolon, fluticasone, fluprednidene, halcinonide, hydrocortisone, momethasone and triamcinolon, and pharmaceutically acceptable esters and acetonides thereof, and mixtures thereof.

The compositions according to the invention principally contain more particularly, as active ingredient, a corticosteroid, preferably clobetasol propionate.

The term “liquid at ambient temperature” means a composition having a viscosity of from 3 to 30 Pa·s (3,000 and 30,000 centipoises), which viscosity is measured with a Brookfield model LVDV II apparatus+spindle No. 4, at a speed of 30 rpm for 30 seconds and at a temperature of 25° C.+/−3° C.

The compositions of the present invention are chemically and physically stable while at the same time allowing good penetration of the active ingredients. Same also have very good acceptability and tolerance among patients, by virtue of its spray formula, as described in the examples to follow of the present invention. It is therefore found that the compositions according to the invention are particularly suitable for the treatment of dermatological afflictions and conditions, and more particularly suitable for the treatment of psoriasis.

This invention therefore features compositions which are liquid at ambient temperature, and which are preferably sprayable, containing, formulated into a pharmaceutically acceptable vehicle:

a) a therapeutically effective amount of clobetasol propionate in solubilized form;

b) an oily phase comprising one or more oils,

such compositions containing no vitamin D derivative, in particular no calcitriol.

Preferably, the compositions according to the invention contain exclusively clobetasol propionate as active ingredient.

The term “solubilized form” means a dispersion of the active agent in the molecular state in a liquid, no crystallization of the active agent being visible to the naked eye nor even under a cross-polarization optical microscope.

The term “composition which is sprayable” means a liquid composition which is fluid and which flows rapidly under its own weight, at ambient temperature. The term “ambient temperature” means a temperature of approximately 25° C.

The spray can be obtained by conventional formulation means known to those skilled in the art, as is explained hereinafter.

Preferably, the composition is anhydrous. For the purpose of the present invention, the term “anhydrous” means a composition substantially free of water, i.e., having a water content of less than or equal to 5% by weight relative to the total weight of the composition, in particular less than or equal to 1%, preferably equal to zero.

Advantageously, the compositions according to the invention comprise from 0.0001% to 0.1% by weight, relative to the total weight of the composition, of a corticoid, preferably from 0.001% to 0.05% by weight. The preferred compositions according to the invention comprise more particularly 0.01%, 0.025% or 0.05% of clobetasol propionate by weight relative to the total weight of the composition.

According to the invention, the term “oily phase” means an oily phase suitable for a pharmaceutical composition.

The oily form is ideal for the psoriasis pathology and approximates a cosmetic massage oil. This liquid oily form makes it possible to give the patient the comfort of emollience without the drawbacks of the application of a thick, very tacky and greasy ointment.

The choice and the ratio of the mixture of oils are made according to their capacities for being spread, their chemical qualities and their capacity as a solvent for the active ingredient. The choice of oils that can be used according to the invention will be made such that the mixture thereof is clear and stable over time.

The predominant oil of the oily phase according to the present invention plays, inter alia, the role of active agent solubilizing agent (also called active agent solvent). The active agent is entirely solubilized in the predominant oil. The oils are the only active agent solvents that can be employed according to the present invention. Thus, alcoholic and glycolic solvents are in particular excluded from the present invention.

According to the invention, the term “predominant oil” means an oil present at a percentage of greater than or equal to 50% by weight relative to the total weight of the oily phase of the composition.

The amount of solvent oil to be introduced into the composition will be defined by the amount of active agent to be solubilized. To this end and as shown by the examples of the present invention, the solubility of the active agent was tested in various oils that can be incorporated in the compositions according to the invention.

The oily phase of the compositions according to the invention may comprise, for example, plant, mineral, animal or synthetic oils, silicone oils, and mixtures thereof.

Exemplary mineral oils include liquid paraffins and various viscosities, such as PRIMOL 352, MARCOL 82 or MARCOL 152 marketed by Esso.

Exemplary plant oils include sweet almond oil, palm oil, soybean oil, sesame oil and sunflower oil.

Exemplary animal oils include lanoline oil, squalene, fish oil and mink oil.

Exemplary synthetic oils include octyldodecanol, an ester such as cetearyl isononanoate, for instance the product marketed under the trademark CETIOL SN by Cognis France, diisopropyl adipate, for instance the product marketed under the trademark CERAPHYL 230 by ISF, isopropyl palmitate, for instance the product marketed under the trademark CRODAMOL IPP by Croda, isononyl isononanoate, such as Dub Inin from the company Stréarinerie Dubois, and caprylic capric triglyceride such as MIGLYOL 812 marketed by Huls/Lambert Riviére, dioctyl ether, such as CETIOL OE marketed by Cognis France, PPG-15 stearyl ether, such as ARLAMOL E marketed by Uniqema; ethyl oleate, such as CRODAMOL OE marketed by Croda.

Exemplary silicone oils include a dimethicone such as the product marketed under the trademark DOW CORNING 200 fluid or Q7 9120 by DOW CORNING, a cyclomethicone such as the product marketed under the trademark Dow Corning 244 fluid by Dow Corning or the product marketed under the trademark MIRASIL CM5 by SACI-CFPA. Also exemplary are volatile silicone oils such as linear siloxanes, and more preferably hexamethyldisiloxane. One example is the product marketed by Dow Corning, DC Fluid 0.65 cSt.

Preferably, the compounds constituting the oily phase of the compositions according to the invention are caprylic/capric triglycerides, marketed under the trademark MIGLYOL 812, cetearyl isononanoate, marketed under the trademark CETIOL SN, cyclomethicones, such as the cyclomethicone 5 marketed under the trademark MIRASIL CM5, dimethicones such as dimethicone 200 having a viscosity of 20 cst, sweet almond oil, ethyl oleate, dioctyl ether and PPG-15 stearyl ether, used alone or a mixture.

The reasons for the selection of these preferred compounds are the following:

Choice of Caprylic/Capric Triglycerides:

Triglycerides are one of the components of the skin; they form part of the natural lipids of the skin with ceramides, cholesterol and phospholipids. They integrate into the deep layers of the epidermis and compensate for the loss of moisturization of the skin. The protective barrier of the skin is regenerated specifically and in a long-lasting manner.

Medium-chain triglycerides, to which the MIGLYOL 812 belongs, are constituted of caprylic (C8) and capric (C10) fatty acids derived from coconut oil or palm kernel oil. Medium-chain triglycerides, to which the MIGLYOL 812 belongs, are constituted of caprylic (C8) and capric (C10) fatty acids derived from coconut oil or palm kernel oil.

The main properties thereof are:

low-viscosity emollient, increasing spreading on the skin;

lipophilic active agent solvent, rapidly penetrates into the skin and promotes penetration of active agent;

no greasy feeling when applied, does not leave any greasy residues.

Choice of Cetearyl Isononanoate:

Cetearyl isononanoate is an ester which has the characteristic of feeling dry and soft on the skin.

Choice of Cyclomethicone 5:

Cyclomethicone 5 is a volatile silicone oil which allows easy application to the skin and leaves a relatively dry feeling after application.

Choice of Dimethicone 200 Having a Viscosity of 20 cst:

The dimethicone is a silicone oil which allows easy application to the skin, avoids the soaping of fatty substances and leaves a non-greasy feeling to the touch after application.

Choice of Sweet Almond Oil:

Sweet almond oil is a plant oil employed for its emollient properties.

Choice of Dioctyl Ether and of PPG-15 Stearyl Ether:

These two ethers have been selected preferentially because they are good solvents for the active ingredient. They are also good emollients and leave a pleasant dry feel.

The mixing and the judicious choice of the oils make it possible to obtain a product that is completely oily but much less greasy and tacky than salves or ointments.

This also allows the patient to apply the product in spray form and allows possible massaging of the region to be treated, unlike a very volatile spray product.

Advantageously, the compositions according to the invention contain from 50% to 99% by weight, relative to the total weight, of oily phase, preferably from 70% to 99% by weight, and more preferably from 85% to 99% by weight.

The present invention therefore features sprayable compositions containing, formulated into a pharmaceutically acceptable vehicle:

a) from 0.0001% to 0.1% of clobetasol propionate;

b) from 50% to 99.9999% of an oily phase comprising one or more oils, selected from among caprylic/capric triglycerides, cetearyl isononanoate, ethyl oleate, PPG-15 stearyl ether, dioctyl ether, cyclomethicones, dimethicones and sweet almond oil, such compositions containing no vitamin D derivative.

More particularly, the sprayable compositions which are preferred according to the present invention contain, formulated into a pharmaceutically acceptable vehicle:

a) from 0.001% to 0.05% of clobetasol propionate;

b) from 85% to 99.999% of an oily phase comprising one or more oils, selected from among caprylic/capric triglycerides, cetearyl isononanoate, ethyl oleate, PPG-15 stearyl ether, dioctyl ether, cyclomethicones, dimethicones and sweet almond oil, such compositions containing no vitamin D derivative.

The compositions according to the invention may also contain surfactants. The surfactants that can be used according to the invention are of the anionic surfactant type, such as carboxylates, and in particular soaps, alkylaryl sulfonates, alkyl ether sulfates, alkyl sulfates or alcohol sulfates. More particularly, the anions of these surfactants are coupled to a cation such as the metal cations of sodium or of potassium. The surfactants which are preferred according to the invention are also surfactants of polysorbate and polyoxamer type.

Preferably, the surfactants according to the present invention are sodium lauryl sulfate, polysorbate 80 (TWEEN 80 from the company Uniquema) and polyoxamer 124 (SYNPERONIC PEL44 from the company Uniquema).

Preferably, the compositions according to the invention also contain propenetrating agents. The propenetrating agents that can be employed according to the invention are of the alcohol type, such as ethanol, or the glycol type, such as 1,2-propanediol, known as propylene glycol, marketed by Dow Chemical, or the dimethyl isosorbide-type, such as ARLASOLVE DMI marketed by Uniqema, the ethoxydiglycol marketed under the trademark TRANSCUTOL HP by Gattefosse, the oleyl alcohol marketed under the trademark HD EUTANOL V PH by Cognis, the n-methyl-2-pyrrolidone marketed under the trademark PHARMASOLVE by ISP, and mixtures thereof.

The propenetrating agent is advantageously selected from among fatty acids and esters thereof, such as, in particular, myristyl lactate, the PEG8 caprylic/capric glycerides marketed under the trademark LABRASOL by Gattefosse, the oleic acid marketed under the trademark OLEINE V2 by Stearinerie Dubois, the propylene glycol monolaurate marketed under the trademark LAUROGLYCOL FCC by Gattefosse, and mixtures thereof.

Preferably, the propenetrating agents used are different from propylene glycol. In fact, the presence of these propenetrating agents makes it possible to formulate the corticoid in anhydrous propenetrating formulations without using propylene glycol.

Even more preferably, the propenetrating agents are selected from among myristyl lactate, dimethyl isosorbide, N-methyl-2-pyrrolidone, PEG8 caprylic/capric glycerides and oleic acid. Even more preferably, the propenetrating agent is myristyl lactate. Advantageously, when this propenetrating agent is used, at least one oil of the composition is cetearyl isononanoate.

The pharmaceutical composition according to the invention may also contain inert additives or combinations of these additives, such as:

wetting agents;

odor improvers;

preservatives;

stabilizing agents;

moisture regulators;

pH regulators;

osmotic pressure modifiers;

emulsifiers;

UV-A and UV-B screens;

propenetrating agents;

chelating agents;

antioxidants;

and synthetic polymers.

Of course, one skilled in the art will take care to select the possible compound(s) to be added to these compositions in such a manner that the advantageous properties intrinsically associated with the present invention are not or not substantially impaired by the addition envisaged.

The compositions according to the invention are more particularly useful for treating the skin and the mucous membranes and are sprayable and suitable for packaging in the sprayable form.

The spray has many advantages compared with conventional forms, such as the ease with which the formula can be delivered to the areas of the body that are very difficult to treat, the possibility of readily controlling the dose delivered or the absence of contamination during use.

The compositions according to the invention are therefore administered in the form of a sprayable composition. The latter can be obtained by conventional formulation means known to those skilled in the art. For example, the composition can be sprayed by means of a mechanical spray device which pumps the composition in a container, bottle or equivalent. Similarly, the composition may be propelled by means of a gas, as is well known by those skilled in the art. Conventional propellant gases such as air or hydrocarbons are utilized provided that they do not interfere with the composition. The composition passes through a nozzle which can be directed directly to the desired site of application. The nozzle can be selected so as to apply the composition in the form of a vaporization or of a jet of droplets, according to the techniques known to those skilled in the art. According to the active pharmaceutical agent selected, the spray mechanism should be capable of always delivering the same amount of active agent. The mechanisms for controlling the amount of composition to be delivered by the spray are also known to those skilled in the art. For example, the amount of propellant gas can be calculated so as to propel the exact amount of product desired. For the composition according to the invention, a metering vaporizer bottle, the application surface area and dose characteristics of which are controlled and reproducible, can be used. For example, the spray device may be constituted of a bottle fitted with a metering valve.

The compositions of the present invention are chemically and physically stable while allowing good penetration of the active ingredient. Same also exhibit very good acceptability and tolerance among the patients, by virtue of its spray formula, as described in the examples of the present invention. It is therefore found that the compositions according to the invention are particularly suitable for the treatment of dermatological conditions, whether regime or regimen.

The present invention therefore features formulating the subject compositions into medicaments useful for the treatment:

of dermatological conditions or afflictions linked to a keratinization disorder relating to differentiation and to proliferation, in particular acne vulgaris, comedone-type acne, polymorphic acne, acne rosacea, nodulocystic acne, acne conglobata, senile acne, secondary acne such as solar acne, acne medicamentosa or occupational acne, of ichthyoses, ichthyosiform states, Darier's disease, palmoplantar keratodermia, leukoplakia and leukoplakia-like states, skin or mucosal (buccal) lichen;

of dermatological conditions or afflictions with an inflammatory immunoallergic component, with or without a cell proliferation problem, in particular cutaneous, mucosal or ungual psoriasis, psoriatic arthritis, skin atopy, such as eczema, respiratory atopy or gingival hypertrophy,

of benign or malignant, dermal or epidermal proliferations of viral or non-viral origin, in particular verruca vulgaris, verruca planar, epidermodysplasia verruciformis, oral or florid papillomatosis, T lymphoma,

of proliferations that may be induced by ultraviolet radiation, in particular basocellular and spinocellular epithelioma,

of precancerous skin lesions, in particular keratoacanthomas,

of immune dermatoses, in particular lupus erythematosus,

of bullous immune diseases,

of collagen diseases, in particular scleroderma,

of dermatological or general conditions or afflictions with immunological components,

of skin disorders caused by exposure to UV radiation, photoinduced or chronological aging of the skin or actinic keratoses or pigmentations, or any pathologies associated with chronological or actinic aging, in particular xerosis,

of sebaceous function disorders, in particular hyperseborrhea of acne, simple seborrhea or seborrhea dermatitis,

of cicatrization disorders or stretch marks,

of pigmentation disorders, such as hyperpigmentation, melasma, hypopigmentation or vitiligo,

of lipid metabolism conditions or disorders, such as obesity, hyperlipidemia, non-insulin-dependent diabetes or syndrome X,

of inflammatory conditions or disorders such as arthritis,

of cancerous or precancerous states,

of alopecia of various origins, in particular alopecia caused by chemotherapy or by radiation,

of immune system disorders, such as asthma, type I diabetes mellitus, multiple sclerosis, or other selective disfunctions of the immune system, or of cardiovascular system conditions or disorders such as arteriosclerosis or hypertension.

In a preferred embodiment of the subject compositions, these will contain 0.0003% of clobetasol propionate and will be used for the production of a medicament useful in treating psoriasis.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Method for the Formulation of the Compositions According to the Invention

The formulation of the compositions according to the present invention is carried out at ambient temperature, under a fume hood and in inactinic light.

The solvent oil is introduced into a flask.

Stirring is begun and then the clobetasol propionate is added.

The stirring is continued until the clobetasol propionate has dissolved.

When the active agent has completely dissolved, the remainder of the constituents of the formula are successively introduced.

The mixture is maintained under stirring until it is completely homogeneous.

### Example 2

| CONSTITUENTS | % |
|---|---|
| CYCLOMETHICONE 5 | QS 100 |
| MEDIUM-CHAIN TRIGLYCERIDES | 40 |
| CLOBETASOL 17-PROPIONATE | 0.025 |
| 1,2-PROPANEDIOL | 10 |
| SWEET ALMOND OIL | 5 |

The protocol is that described in Example 1.

A very slightly yellow, liquid solution is obtained.

### Example 3

| CONSTITUENTS | % |
|---|---|
| CETEARYL ISONONANOATE | QS 100 |
| MEDIUM-CHAIN TRIGLYCERIDE | 40 |
| CLOBETASOL 17-PROPIONATE | 0.025 |
| DIMETHICONE, 20 CST | 10 |
| SWEET ALMOND OIL | 5 |

The protocol is that described in Example 1.

A very slightly yellow liquid solution is obtained.

### Example 4

| CONSTITUENTS | % |
|---|---|
| MEDIUM-CHAIN TRIGLYCERIDES | QS 100 |
| CYCLOMETHICONE 5 | 45 |
| CLOBETASOL 17-PROPIONATE | 0.025 |
| DIMETHICONE, 20 CST | 10 |
| SWEET ALMOND OIL | 5 |

The protocol is that described in example 2.

A very slightly yellow liquid solution is obtained.

### Example 5

| CONSTITUENTS | % |
|---|---|
| MEDIUM-CHAIN TRIGLYCERIDES | QS 100 |
| CYCLOMETHICONE 5 | 35 |
| CLOBETASOL 17-PROPIONATE | 0.025 |
| DIMETHICONE, 20 CST | 10 |
| SWEET ALMOND OIL | 5 |

The protocol is that described in Example 1.

A very slightly yellow liquid solution is obtained.

### Example 6

| CONSTITUENTS | % |
|---|---|
| CETEARYL ISONONANOATE | QS 100 |
| CYCLOMETHICONE 5 | 35 |
| CLOBETASOL 17-PROPIONATE | 0.05 |
| PROPYLENE GLYCOL | 10 |
| SWEET ALMOND OIL | 5 |
| PEG8 CAPRYLIC/CAPRIC | 2 |
| GLYCERIDES | |

The protocol is that described in Example 1.

A very slightly yellow liquid solution is obtained.

### Example 7

| CONSTITUENTS | % |
|---|---|
| DIISOPROPYL ADIPATE | 30 |
| CYCLOMETHICONE 5 | QS 100 |
| CLOBETASOL 17-PROPIONATE | 0.025 |
| ETHOXYDIGLYCOL | 1.50 |
| OLEIC ACID | 2 |
| SWEET ALMOND OIL | 5 |

The protocol is that described in Example 1.

A very slightly yellow liquid solution is obtained.

### Example 8

| CONSTITUENTS | % |
|---|---|
| MINERAL OIL | QS 100 |
| CYCLOMETHICONE 5 | 30 |
| CLOBETASOL 17-PROPIONATE | 0.01 |
| POLYSORBATE 80 | 1 |
| PROPYLENE GLYCOL | 10 |
| MONOLAURATE | |
| DIMETHICONE, 20 cst | 10 |

The protocol is that described in Example 1.

An opaque liquid solution is obtained.

### Example 9

| CONSTITUENTS | % |
|---|---|
| ETHYL OLEATE | 20 |
| CETEARYL ISONONANOATE | QS 100 |
| CLOBETASOL 17-PROPIONATE | 0.05 |
| ETHANOL | 3.50 |
| N-METHYL-2-PYRROLIDONE | 0.8% |
| SWEET ALMOND OIL | 5 |

The protocol is that described in Example 1.

A slightly yellow liquid solution is obtained.

### Example 10

| CONSTITUENTS | % |
|---|---|
| CLOBETASOL 17-PROPIONATE | 0.05% |
| BUTYLHYDROXYTOLUENE | 0.04% |
| MEDIUM-CHAIN TRIGLYCERIDES | 49.91% |
| Cyclomethicone 5 | 35.00% |
| Dimethicone, 20 CST | 10.00% |
| SWEET ALMOND OIL | 5.00% |

The protocol is that described in Example 1.

### Example 11

| CONSTITUENTS | % |
|---|---|
| MYRISTYL LACTATE | 5 |
| CETEARYL ISONONANOATE | QS 100 |
| CLOBETASOL 17-PROPIONATE | 0.05 |
| CYCLOMETHICONE 5 | 35 |
| ETHANOL | 3.50 |
| DIMETHICONE, 20 cst | 10 |
| SWEET ALMOND OIL | 5 |

The protocol is that described in Example 1.

A slightly yellow liquid solution is obtained,

### Example 12

| CONSTITUENTS | % |
|---|---|
| MYRISTYL LACTATE | 2 |
| MEDIUM-CHAIN TRIGLYCERIDES | QS 100 |
| CYCLOMETHICONE 5 | 45 |
| CLOBETASOL 17-PROPIONATE | 0.025 |
| DIMETHICONE, 20 CST | 10 |
| SWEET ALMOND OIL | 5 |

The protocol is that described in Example 1.

A slightly yellow liquid solution is obtained.

### Example 13

Maximum solubility of clobetasol propionate:

The maximum solubility of clobetasol propionate was studied in various solvent oils according to the invention, in order to allow the choice most suitable for the concentration of active agent to be solubilized.

It is measured by the UV assaying method.

Saturated solutions are prepared according to the following protocol:

0.1% of clobetasol propionate is added to the oily excipient.

The entire mixture is stirred overnight and then left for two hours without stirring.

The solutions for UV-assaying are then prepared by taking the supernatant 0.1 g of this saturated solution is weighed into a 100 ml volumetric flask and then made up to 100 ml with absolute ethanol. The assaying makes it possible to obtain data for maximum solubility of the active agent in the solvent oil under consideration.

The solubility can also be assessed visually. Thus, minute proportions of the active agent are gradually added to the solvent oil with stirring. Visual observation of the clarity of the solution is carried out. The appearance of cloudiness means that the maximum percentage of active agent to be incorporated has been reached, hence deduction of a maximum solubility value.

Result:

| | | Visual |
|---|---|---|
| Solvent employed | UV Solubility at 266 nm | solubility |
| Isopropyl palmitate | 0.17% | 0.14% |
| Caprylic/capric | not determined | 0.25% |
| triglycerides | | |
| Octyldodecanol | not determined | 0.16% |
| Dioctyl ether | 0.12% | insoluble |
| Oleyl alcohol | 0.86% | / |
| Ethyl oleate | 0.79% | / |
| Oleic acid | 0.45% | / |
| Cetearyl | 0.16% | / |
| isononanoate | | |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A pharmaceutical/dermatological composition which comprises, formulated into a pharmaceutically acceptable vehicle:
a) a therapeutically effective amount of a corticoid solubilized therein; and
b) an oily phase comprising one or more oils;
said composition containing no calcitriol; said composition being liquid at ambient temperature and physically and chemically stable.

2. The pharmaceutical/dermatological composition as defined by claim 1, formulated in sprayable form.

3. The pharmaceutical/dermatological composition as defined by claim 1, wherein the corticoid is solubilized in said oily phase.

4. The pharmaceutical/dermatological composition as defined by claim 1, wherein the corticoid is clobetasol propionate.

5. The pharmaceutical/dermatological composition as defined by claim 1, wherein the oily phase contains one or more oils selected from the group consisting of caprylic/capric triglycerides, cetearyl isononanoate, ethyl oleate, cyclomethicones, dimethicones, sweet almond oil, dioctyl ether and PPG-15 stearyl ether.

6. The pharmaceutical/dermatological composition as defined by claim 1, wherein said pharmaceutically acceptable vehicle contains:
a) from 0.0001% to 0.1% of clobetasol propionate;
b) from 50% to 99.9999% of an oily phase comprising one or more oils selected from among caprylic/capric triglycerides, cetearyl isononanoate, ethyl oleate, PPG-15 stearyl ether, dioctyl ether, cyclomethicones, dimethicones and sweet almond oil, and said composition containing no calcitriol.

7. The pharmaceutical/dermatological composition as defined by claim 1, wherein said pharmaceutically acceptable vehicle contains:
a) from 0.001% to 0.05% of clobetasol propionate;
b) from 85% to 99.999% of an oily phase comprising one or more oils selected from among caprylic/capric triglycerides, cetearyl isononanoate, ethyl oleate, PPG-15 stearyl ether, dioctyl ether, cyclomethicones, dimethicones and sweet almond oil, and said composition containing no calcitriol.

8. The pharmaceutical/dermatological composition as defined by claim 1, further comprising a surfactant compound.

9. The pharmaceutical/dermatological composition as defined by claim 8, wherein said surfactant is selected from among sodium lauryl sulfate, poloxamers and polysorbates.

10. The pharmaceutical/dermatological composition as defined by claim 1, further comprising a propenetrating compound.

11. The pharmaceutical/dermatological composition as defined by claim 10, wherein said propenetrating agent is selected from among ethanol, 1,2-propanediol, dimethyl isosorbide, ethoxydiglycol, PEG8 caprylic/capric glycerides, oleic acid, propylene glycol monolaurate, N-methyl-2-pyrrolidone, oleyl alcohol and myristyl lactate.

12. A regime or regimen for the treatment:
of dermatological conditions or afflictions linked to a keratinization disorder relating to differentiation and to proliferation, acne vulgaris, comedone-type acne, polymorphic acne, acne rosacea, nodulocystic acne, acne conglobata, senile acne, secondary acne, solar acne, acne medicamentosa or occupational acne,
of ichthyoses, ichthyosiform states, Darier's disease, palmoplantar keratodermia, leukoplakia and leukoplakia-like states, skin or mucosal (buccal) lichen;
of dermatological conditions or afflictions with an inflammatory immunoallergic component, with or without a cell proliferation problem, cutaneous, mucosal or ungual psoriasis, psoriatic arthritis, skin atopy, eczema, respiratory atopy or gingival hypertrophy,
of benign or malignant, dermal or epidermal proliferations of viral or nonviral origin, verruca vulgaris, verruca planar, epidermodysplasia verruciformis, oral or florid papillomatosis, T lymphoma,
of proliferations induced by ultraviolet radiation, basocellular and spinocellular epithelioma,
of precancerous skin lesions, keratoacanthomas,
of immune dermatoses, lupus erythematosus,
of bullous immune diseases,
of collagen diseases, scleroderma,
of dermatological or general conditions or afflictions with immunological component,
of skin disorders caused by exposure to UV radiation, photoinduced or chronological aging of the skin or actinic keratoses or pigmentations, or any pathologies associated with chronological or actinic aging, xerosis,
of sebaceous function disorders, hyperseborrhea of acne, simple seborrhea or seborrhea dermatitis,
of cicatrization disorders or stretch marks,
of pigmentation disorders, hyperpigmentation, melasma, hypopigmentation or vitiligo,
of lipid metabolism conditions, obesity, hyperlipidemia, non-insulin-dependent diabetes or syndrome X,
of inflammatory conditions, arthritis,
of cancerous or precancerous states,
of alopecia of various origins, alopecia caused by chemotherapy or by radiation,
of immune system disorders, asthma, type I diabetes mellitus, multiple sclerosis, or other selective disfunctions of the immune system, or
of cardiovascular system conditions or disorders, arteriosclerosis or hypertension, comprising administering to an individual in need of such treatment, a thus effective amount of the pharmaceutical/dermatological composition as defined by claim 1.

13. A regime or regimen for the treatment of psoriasis, comprising administering to an individual in need of such treatment, a thus effective amount of the pharmaceutical/dermatological composition as defined by claim 1.

14. A regime or regimen for the treatment of psoriasis, comprising spraying onto the affected skin area of an individual in need of such treatment, a thus effective amount of the pharmaceutical/dermatological composition as defined by claim 1.

15. The pharmaceutical/dermatological composition as defined by claim 1, comprising a corticoid selected from the group consisting of betamethasone clobetasol, clobetasone, desoximethasone, diflucortolon, diflorasone, fluocinonide, flumethasone, fluocinolon, fluticasone, fluprednidene, halcinonide, hydrocortisone, momethasone and triamcinolon, and pharmaceutically acceptable esters, acetonides and mixtures thereof.

16. A pharmaceutical/dermatological composition which comprises, formulated into a pharmaceutically acceptable vehicle:
a) a therapeutically effective amount of a corticoid solubilized therein; and
b) an oily phase comprising one or more oils;
said composition containing no calcitriol, alcoholic compounds, or glycolic compounds; said composition being liquid at ambient temperature and physically and chemically stable.
